(19) United States
(12) Patent Application Publication
(10) Pub. No.: US 2021/0393474 A1
(43) Pub. Date: Dec. 23, 2021

(54) Multi-function pelvic trigger point wand and related software.

(71) Applicant: Nicholas Hernan, Del Mar, CA (US)

(72) Inventor: Nicholas Hernan, Del Mar, CA (US)

(21) Appl. No.: 17/349,428

(22) Filed: Jun. 16, 2021

Related U.S. Application Data
(60) Provisional application No. 63/042,081, filed on Jun. 22, 2020.

(51) Int. Cl. A61H 19/00; A61H 23/02

(52) U.S. Cl. CPC A61H 19/44; A61H 23/0263; A61H 2201/5074; A61H 2201/5007; A61H 2201/10; A61H 2201/0207; A61H 2230/60

(57) ABSTRACT

A Multi-function Pelvic Trigger Point Massage Wand is a handheld device for the treatment of pelvic pain and pain/discomfort caused by related urinary and sexual dysfunctions. The device provides an adjustable combination of heat, vibration, electric stimulation, electromyography, photoplethysmography pulse oximetry, and biofeedback, depending on the treatment regimen desired. A metal sensor band around the release-end of the wand conducts electromyography and electrical stimulation. Photodiode sensors are coupled with green and infrared LEDs to calculate the users heart rate and blood flow. A sensor for biofeedback measurement assigns data produced from electrical stimulation sensors, electromyography sensors, photodiode sensors and the biofeedback/pressure measuring function to a smart device with operating software created specifically for the pelvic trigger point wand. The information output is collected and analyzed to show the results of different treatment techniques.

### CROSS REFERENCE TO RELATED APPLICATIONS

This application claims priority to U.S. Provisional No. 63/042,081 filed Jun. 26, 2020, the contents of which are incorporated by reference.

### STATEMENT REGARDING FEDERALLY SPONSORED RESEARCH OR DEVELOPMENT

This invention was not federally sponsored.

### BACKGROUND OF THE INVENTION

### Field of the Invention

Invention relates to the general field of pelvic treatment devices, and more specifically, to the field of therapeutic wands used to treat pelvic disorders and pelvic pain.

This particular invention is an active medical device that combines a customizable combination of heat, vibration, electric muscle stimulation, photoplethysmography, electromyography, pulse oximetry, and biofeedback measurements including those of muscle tension, pressure and sensitivity depending on the treatment regimen desired.

Pelvic pain can originate from a variety of disorders of the female reproductive system. Including but not limited to Adenomyosis, Ectopic pregnancy, Endometriosis, Menstrual cramps, Miscarriage (before the 20th week) or intrauterine fetal death, Mittelschmerz, Ovarian cancer, Ovarian cysts, Pelvic inflammatory disease, Uterine fibroids, and Vulvodynia. Other causes in women and men include Appendicitis, Colon cancer, Constipation, Crohn's disease (a type of inflammatory bowel disease), Diverticulitis, Fibromyalgia, Inguinal hernia, Interstitial cystitis (also called painful bladder syndrome), Intestinal obstruction, Irritable bowel syndrome, Kidney stones, Past physical or sexual abuse, Pelvic floor muscle spasms, Prostatitis, Ulcerative colitis, and Urinary tract infection (UTI). Pelvic pain can also originate in men from a variety of disorders including but not limited to: Appendicitis, Colon cancer, Constipation, Crohn's disease, Diverticulitis, Fibromyalgia, Inguinal hernia, Interstitial cystitis, Intestinal obstruction, Irritable bowel syndrome, Kidney stones, Past physical or sexual abuse, Pelvic floor muscle spasms, Prostatitis, Ulcerative colitis, and Urinary tract infections.

### Brief Description of the Problem Solved

Previously, resources focused on treating pelvic pain typically have been focused on psychiatrics, biological originations, nerve specific treatment, surgery and autoimmune disorders. Research has shown that that pain can be treated by focusing on the tension located in the pelvic floor muscles, commonly referred to as trigger points. Prior to the advent of pelvic therapy wands, physical therapists, gynecologists and other healthcare professionals specializing in pelvic therapy had used their fingers and hands to ease tight muscles, trigger points, and discomfort. The creation of pelvic therapy wands allowed practitioners to more easily access areas of applied treatment than what was possible before. In addition, pelvic wands provided patients with a self-treatment options that alternative methods were incapable of either due to cost, knowledge barriers, or apprehension and anxiety concerning various treatments. These more accessible treatments include external and internal self-massage and myofascial release within the pelvic musculature.

The field of pelvic therapy wands has a small number of distinctly-shaped wands, each for a different function, and while there exist devices that provide heat, vibrate, and even receive biofeedback, there is not a device that combines all these factors into a single wand. Since humans in need of pelvic care are different in terms of what the optional treatment for each person would be, it is desirable to have a single wand that could be used for different people who need different treatments, and potentially provide different approaches to treatment as that person's needs may change over time. It is also beneficial to provide a wand that can assess the treatments being given, and synthesize a new treatment regimen based upon biofeedback from the patient. Further, the quantified self-treatment may help both the patient and their provider discover insights which could benefit the patient in terms of more effective and specialized care.

### PRIOR ART

The prior art contains a variety of single-function wands and methods of treating pelvic pain that require much larger or complicated devices that rely on use of external biofeedback “machines” to collect and process the data. For example, U.S. Pat. Nos. 8,337,435, 8,639,360 and 8,224,464 to Wise provide an internal treatment device the provides massage capabilities, but lacks the other benefits provided by this invention. Indeed, U.S. Pat. No. 8,337,435 only applies manual pressure to the trigger points while U.S. Pat. Nos. 8,639,360 and 8,224,464 provide a biofeedback component which depends upon a series of outside wires to connect to the unit that displays the feedback to user. U.S. Pat. No. 7,628,744 to Anatasol describes a “pelvic exercise probe” which collects biofeedback from the pelvic region but does not provide the other capabilities of this invention. U.S. Pat. No. 9,844,681 to Mcube Technology provides a complicated biofeedback device which, again, fails to contain the other beneficial components of this invention.

The current invention provides a solution by providing a hand-held wand for the treatment of pelvic pain and pain/discomfort caused by associated dysfunctions. The wand is multi-functional, in that it can provide heat, vibration (Utilizing eccentric rotating mass) and haptic feedback (linear resonant actuator), electric muscle stimulation, electromyography, photoplethysmography, pulse oximetry, and forms of biofeedback such as measuring muscle contracting, pressure, and sensitivity in the pelvic floor musculature, all in the same device. The wand incorporates a band around the release-end of the wand that is capable of conducting electromyography and electrical stimulation to aid in and provide detailed measurements of electrical activity in skeletal muscles accounting for different dynamic aspects and responses during the treatment of pelvic floor pain. Photodiode sensors located near the electrical sensors on the release-end that are coupled with green and infrared LEDs that flash hundreds of times per second to calculate the user's heart rate and blood flow. At the tip of the release end are sensors for biofeedback measurements. The data produced from electromyography sensors, photodiode and LEDs and the biofeedback measuring functions are sent via Bluetooth to a smart device containing software created specifically for the pelvic trigger point wand. The information output by the device is collected and analyzed to compile digital animations to show the user how different treatment techniques effect his or her body and to provide quantitatively determined reference points during the progress of treatment.

The software is an important component of the wand. The software analyzes data supplied to it by the biofeedback mechanisms in the wand and generates an optimal treatment plan based on the user's response to the different methods used. As opposed to the software programs of any single function wands, this invention detects abnormalities such as heart rhythm, sensitivity, tissue damage, trigger point location, blood flow, muscle strength and progression of treatment. All of this data us used to generate an individualized treatment plan based on the biofeedback data. Incorporating various technologies into one device capable of performing myofascial release in the pelvic floor musculature eliminates the need for purchases of multiple devices and trips to healthcare practitioners if use of the device(s) at home is not feasible. Using one device instead of multiple reduces risks associated with each of the other devices and will reduce unnecessary costs and other burdens along with increased likelihood of treatment success. The multi-functional device capable of various measurements centralizes data which can relay more accurate and useful information to the user that is not possible when different devices are used for each individual function, especially when these devices are all manufactured with different companies and do not share information critical to successful treatment. A key advantage of this invention is that it is a single device capable of performing multiple functions. Thereby this may reduce treatment costs and eliminates risks of gaps in data produced and analyzed.

This device is meant for at home use which can eliminate any apprehension the patient may have when going to a medical office for treatment. Treatment data can be shared with the healthcare practitioner remotely. This product is rechargeable by induction and does not need battery replacements.

### OBJECTS OF THE INVENTION

It is therefore an object of the present invention to provide a handheld, multi-function wand effective in assessing and aids in providing individualized treatment to the user based on various biofeedback data taken from the patient.

There has thus been outlined, rather broadly, the more important features of the invention in order that the detailed description thereof may be better understood, and in order that the present contribution to the art may be better appreciated. There are additional features of the invention that will be described hereinafter and which will form the subject matter of the claims appended hereto. The features listed herein and other features, aspects and advantages of the present invention will become better understood with reference to the following description and appended claims. The accompanying drawings, which are incorporated in and constitute part of this specification, illustrate embodiments of the invention and, together with the description, serve to explain the principles of the invention.

It should be understood the while the preferred embodiments of the invention are described in some detail herein, the present disclosure is made by way of example only and that variations and changes thereto are possible without departing from the subject matter coming within the scope of the following claims, and a reasonable equivalency thereof, which claims I regard as my invention.

### BRIEF DESCRIPTION OF THE FIGURES

One preferred form of the invention will now be described with reference to the accompanying drawings.

FIG. 1. Is a perspective view of the induction contact point.

FIG. 2 is a perspective view of the power button.

FIG. 3 is a perspective view of the rechargeable battery.

FIG. 4 is a perspective view of the printed circuit board.

FIG. 5 is a perspective view of the heating coil.

FIG. 6 is a perspective view of the eccentric mass motor.

FIG. 7 is a perspective view of the linear resonating actuator.

FIG. 8 is a perspective view of the force resisting load sell/pressure sensor.

FIG. 9 is a perspective view of the photodiodes and colored/Infrared LED Mechanism.

FIG. 10 is a side, perspective view of the multi-function wand.

FIG. 11 is a cut-away perspective drawing of the multi-function wand.

### DETAILED DESCRIPTION OF THE FIGURES

Many aspects of the invention can be better understood with references made to the drawings below. The components in the drawings are not necessarily drawn to scale. Instead, emphasis is placed upon clearly illustrating the components of the present invention. Moreover, like reference numerals designate corresponding parts through the several views in the drawings.

Before explaining at least one embodiment of the invention, it is to be understood that the embodiments of the invention are not limited in their application to the details of construction and to the arrangement of the components set forth in the following description or illustrated in the drawings. The embodiments of the invention are capable of being practiced and carried out in various ways. In addition, the phraseology and terminology employed herein are for the purpose of description and should not be regarded as limiting.

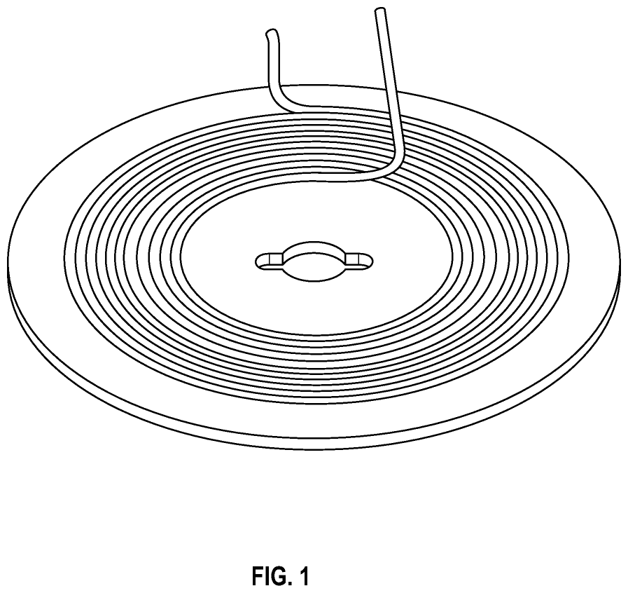

FIG. 1. Is a perspective view of the induction contact point.

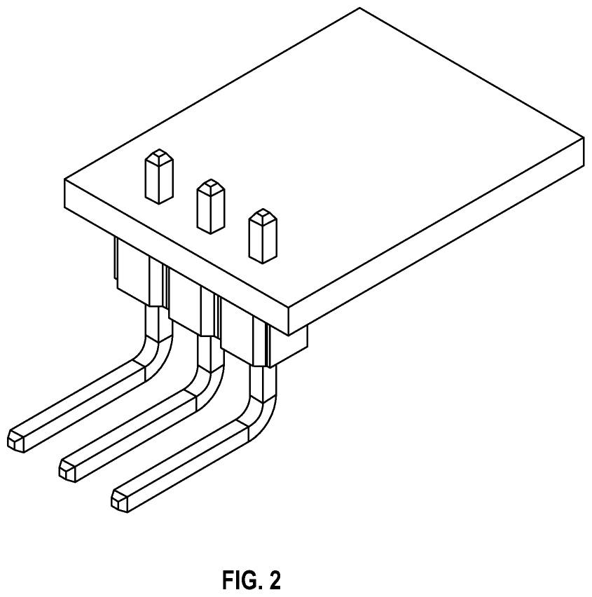

FIG. 2 is a perspective view of the power button.

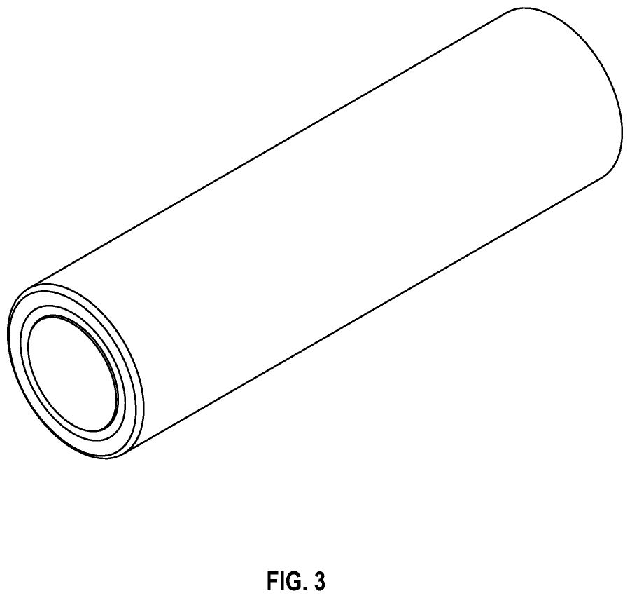

FIG. 3 is a perspective view of the rechargeable battery.

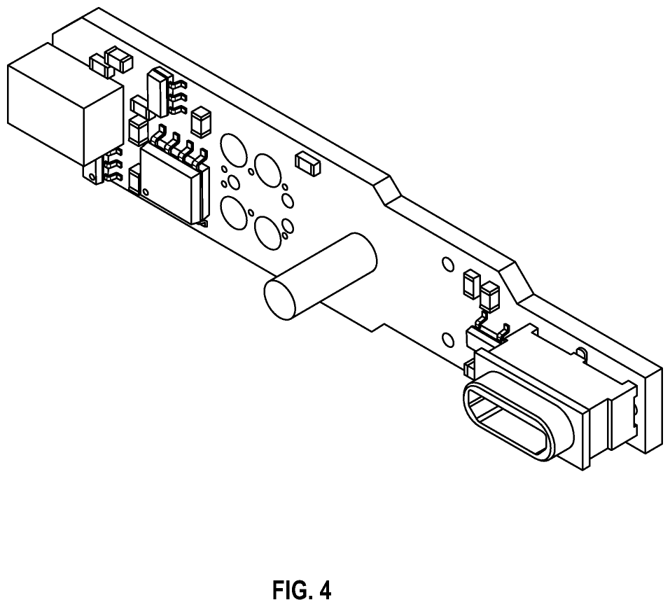

FIG. 4 is a perspective view of the printed circuit board.

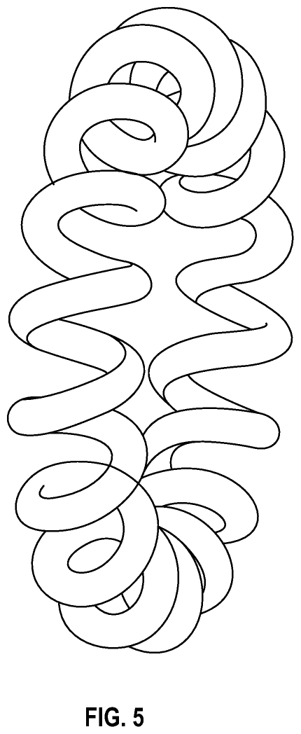

FIG. 5 is a perspective view of the heating coil.

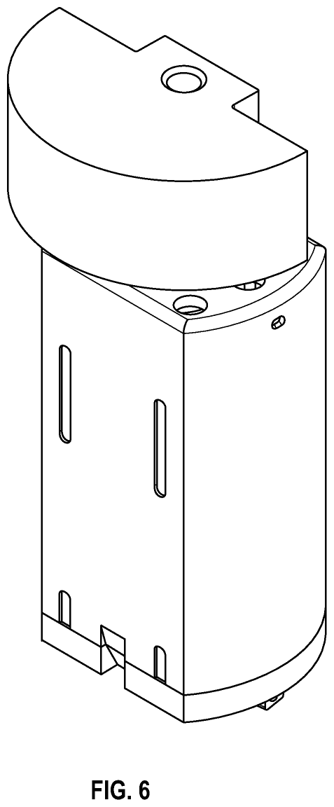

FIG. 6 is a perspective view of the eccentric mass motor.

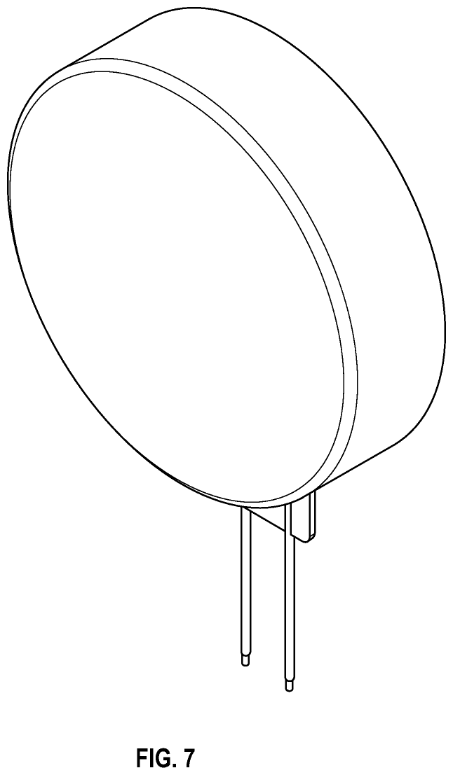

FIG. 7 is a perspective view of the linear resonating actuator.

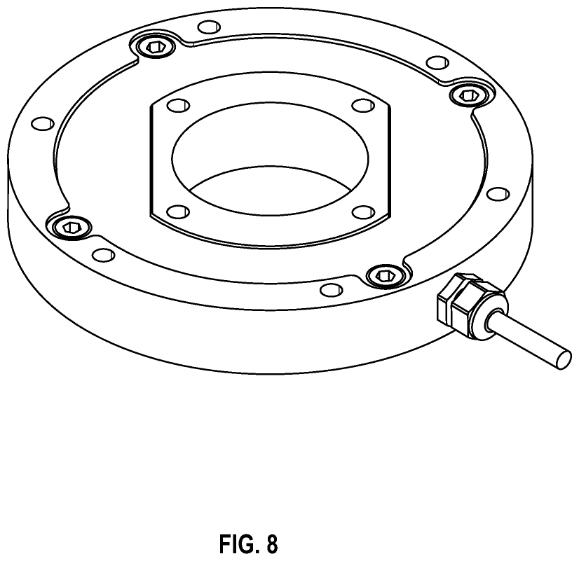

FIG. 8 is a perspective view of the force resisting load sell/pressure sensor.

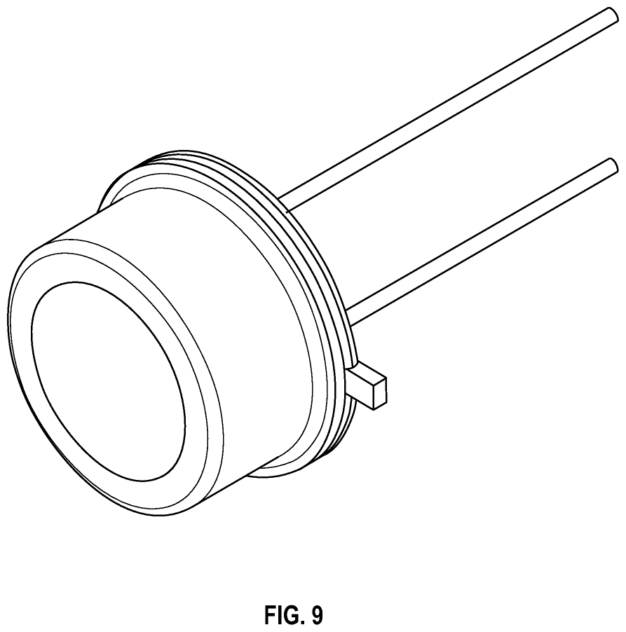

FIG. 9 is a perspective view of the photodiodes and colored/Infrared LED Mechanism.

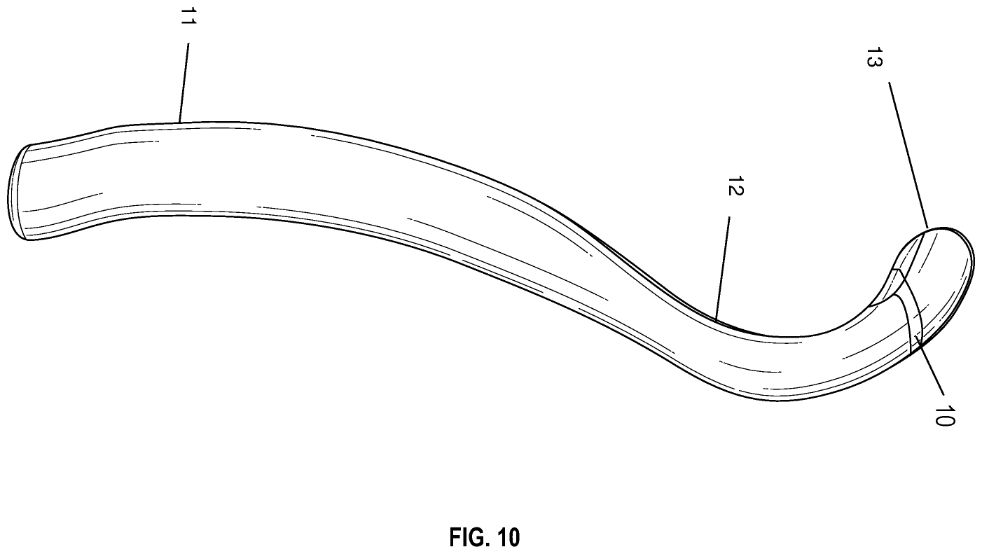

FIG. 10 is a side, perspective view of the multi-function wand. The treatment tip 13 is separated by the neck 12 from the body 11. The neck is of lesser diameter than the body or tip for the comfort of the patient. The Electromyography and electrical muscle stimulation unit 10 is a band on the outside of the body.

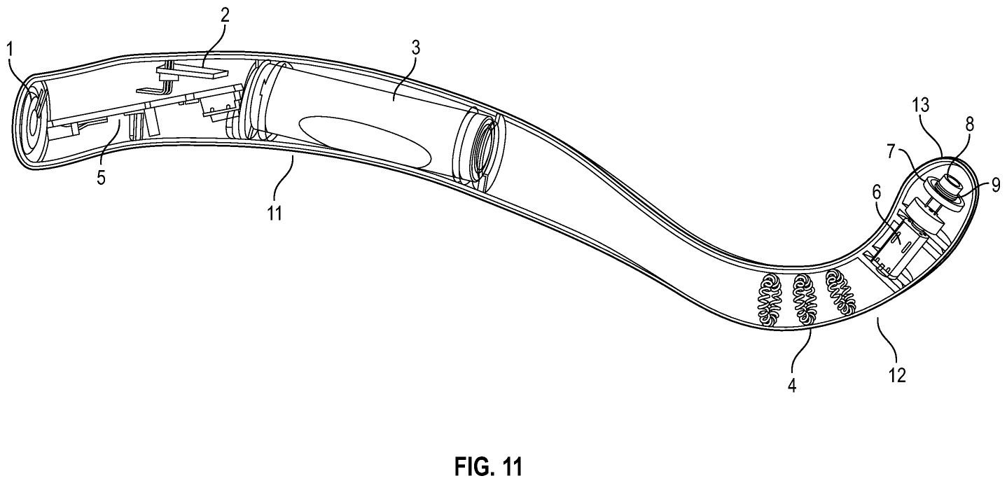

FIG. 11 is a cut-away perspective drawing of the multi-function wand. The induction contact point 1 is used to charge the rechargeable battery 3. A power button 2 allows the user to turn on and off the machine and to adjust the settings of the device. The wand also has a vibratory linear resonating motor 7 on a cam to create vibration when such is a desired treatment. One or more heating coils 4 provide warmth, which a printed circuit board 5 controls the actions of the wand and synthesizes biofeedback from the force resisting load sell (pressure sensor) 8 the Photodiodes and Colored/Infrared LED Mechanism 9 and the Electromyography and electrical muscle stimulation unit 10.

FIG. 2 is a side, perspective view of the multi-function wand. T

In terms of how the invention functions, the Multi-function Pelvic Trigger Point Massage Wand is a small wand that can be held in a person's hand, and provides a number of treatments for pelvic pain and pain/discomfort caused by related urinary and sexual dysfunctions.

The treatment end, or release end, is the part of the wand that is used to apply direct pressure to internal trigger points located in the pelvic musculature. This device can also be used to relieve trigger points on the external areas of the pelvic region in men and women. The treatment end is where the measurement and active technology of the device are located. This includes the force resistors for measuring pressure, along with the LEDs and diodes for measuring blood flow and heart rate. In addition, the electrically conductive materials which pass through to the external areas of the treatment end are used for muscle stimulation and electromyography. Situated behind these measurement features are a haptic feedback motor and an eccentric rotating mass motor for customizable vibration. This vibration is used for treatment and instructive feedback. Wires, not visible in this figure, connect these devices to the printed circuit assembly (PCA) which allows for operation of the device and the relay of information back to a device capable of running the software used with the wand. Also located on the PCA are components for capacitive operation of the device. Located in between the PCA and the treatment functions is a lithium ion battery. At the end of the device opposite to the treatment side are the components for allowing induction charging, otherwise known as wireless charging.

The wand provides a number of functional benefits to a person needing pelvic treatment. It is capable of creating and providing heat, vibration, electric stimulation, electromyography, photoplethysmography, pulse oximetry, and receiving biofeedback from a patient. The release end is the finger-like end of the device that is inserted rectally or vaginally. Located at the base of the release-end of the device are electronic components capable of thermal conduction to warm the device which helps blood flow and relaxation in the pelvic floor during use of the device. The device also incorporates a metal sensor band around the release-end of the wand for conducting electromyography and electrical stimulation to aid in and provide detailed measurement and during the treatment of pelvic floor pain. Photodiode sensors located near the electrical sensors on the release-end are coupled with green and infrared LEDs that flash hundreds of times per second to calculate the users heart rate and blood flow. Near the tip of the release end is are sensors for biofeedback measurements. The data produced from electrical stimulation sensors, electromyography sensors, photodiodes, LEDs and the biofeedback measuring functions are sent via Bluetooth to a smart device containing software created specifically for the pelvic trigger point wand.

The information output by the device is collected and analyzed, along with the device's analysis of the pelvic floor health to compile digital animated graphics to show the user how different treatment techniques effect his or her body, and to detect abnormalities in the pelvic floor. The data generated provides quantitative reference points for a user to track the progress of their treatment.

The software that was developed specifically for this wand uses biofeedback and other data to generate an optimal treatment plan based on the user's response to the different methods used. Incorporating various technologies into one device capable of performing myofascial release in the pelvic floor musculature eliminates the need for purchases of multiple devices and repeat trips to healthcare practitioners if use of the device(s) at home is not feasible. Using one device instead of multiple devices reduces risks associated with each of the other devices. The multi-functional device capable of various measurements centralizes data which can relay more accurate and useful information to the user that is not possible when different devices are used for each individual function, especially when these devices are all manufactured with different companies and do not share information. The fact that this invention provides a single device that can perform multiple functions also eliminates risk of continuity lapses in data produced and analyzed.

This device is meant for at home use which can eliminate any apprehension the patient may have when going to a medical office for treatment. Treatment data can be shared with the healthcare practitioner remotely via software. This product is rechargeable by induction coils and does not need battery replacements.

It should be understood that while the preferred embodiments of the invention are described in some detail herein, the present disclosure is made by way of example only and that variations and changes thereto are possible without departing from the subject matter coming within the scope of the following claims, and a reasonable equivalency thereof, which claims I regard as my invention.

All of the material in this patent document is subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

### REFERENCE NUMBERS USED

1. Induction Contact Point
2. Power Button
3. Rechargeable Battery
4. Printed Circuit Board
5. Temperature Coil
6. Eccentric Mass Motor
7. Linear Resonating Actuator
8. Load Cell
9. Photodiode and LED Mechanism
10. Electromyography and electrical muscle stimulation band
11. Handle/Body
12. Neck
13. Release End

That which is claimed:

1. A multi-function device for treating pelvic disorders, consisting of: a wand and a software element, where the wand comprises a release end, a neck, a body and an end, where the end comprises a charging connection via induction coils at an induction contact point, where the body comprises a printed circuit board, an eccentric mass motor, one or more heating coils, a power button, a linear resonating actuator, a force resisting load sell/pressure sensor, a Photodiodes and Colored/Infrared Mechanism, an Electromyography and electrical muscle stimulation unit and a rechargeable battery, where one or more wires connect induction contact point, the rechargeable battery, the power button, the printed circuit board and the eccentric mass motor, where the release end comprises a force pressure measurement device, and a linear resonating actuator, and a photodiodes couples with green and infrared LED's, where an external portion of the tip retains a Electromyography and electrical muscle stimulation band for conducting electrical stimulation and detecting measurements of activity in the skeletal muscles of the pelvis, through a process of electromyography, and where the software element comprises a biofeedback input, a data synthesis component, and a predictive analysis component, where the software element receives the biofeedback element, synthesizes the biofeedback element, and synthesizes a revised treatment plan based on the synthesis of the biofeedback element.

2. The device of claim 1, where the number of heating coils is three.

3. The device of claim 2, where the Electromyography and electrical muscle stimulation band extends from an inside of the wand to an outer surface of the wand.

4. A multi-function device for treating pelvic disorders, comprising: a wand and a software element, where the wand comprises a release end, a neck, a body and an end, where an external portion of the tip retains a Electromyography and electrical muscle stimulation band for conducting electrical stimulation and detecting measurements of activity in the skeletal muscles of the pelvis.

5. The device of claim 4, where the software element comprises a biofeedback input, a data synthesis component, and a predictive analysis component, where the software element receives the biofeedback element, synthesizes the biofeedback element, and synthesizes a revised treatment plan based on the synthesis of the biofeedback element.

6. The device of claim 5, where the end comprises a charging connection via induction coils at an induction contact point, where the body comprises a printed circuit board, an eccentric mass motor, one or more heating coils, a power button, a linear resonating actuator, a force resisting load sell/pressure sensor, a Photodiodes and Colored/Infrared Mechanism, an Electromyography and electrical muscle stimulation unit and a rechargeable battery, where one or more wires connect induction contact point, the rechargeable battery, the power button, the printed circuit board and the eccentric mass motor.

7. The device of claim 6, where the release end comprises a force pressure measurement device, and a linear resonating actuator.

8. The device of claim 7, where the release end additionally comprises a photodiodes couples with green and infrared LED's,

9. The device of claim 8, where an external portion of the tip retains a Electromyography and electrical muscle stimulation band for conducting electrical stimulation and detecting measurements of activity in the skeletal muscles of the pelvis.

10. The device of claim 9, where a biofeedback from a patient is measure through electromyography.

11. The device of claim 10, where the number of heating coils is three.

12. The device of claim 11, where the Electromyography and electrical muscle stimulation band extends from an inside of the wand to an outer surface of the wand.

13. The device of claim 11, where the Electromyography and electrical muscle stimulation band provides muscle stimulation and obtains biofeedback from the patient.

14. A multi-function device for treating pelvic disorders, comprising: a wand and a software element, where the wand comprises a release end, a neck, a body and an end, where an external portion of the tip retains a Electromyography and electrical muscle stimulation band for conducting electrical stimulation and detecting measurements of activity in the skeletal muscles of the pelvis.

15. The device of claim 14, where the end comprises a charging connection via induction coils at an induction contact point, where the body comprises a printed circuit board, an eccentric mass motor, one or more heating coils, a power button, a linear resonating actuator, a force resisting load sell/pressure sensor, a Photodiodes and Colored/Infrared Mechanism, an Electromyography and electrical muscle stimulation unit and a rechargeable battery, where one or more wires connect induction contact point, the rechargeable battery, the power button, the printed circuit board and the eccentric mass motor.

16. The device of claim 15, where the release end comprises a force pressure measurement device, and a linear resonating actuator, where an external portion of the tip retains a Electromyography and electrical muscle stimulation band for conducting electrical stimulation and detecting measurements of activity in the skeletal muscles of the pelvis.

17. The device of claim 16, where the release end additionally comprises a photodiodes couples with green and infrared LED's, where the software element comprises a biofeedback input, a data synthesis component, and a predictive analysis component, where the software element receives the biofeedback element, synthesizes the biofeedback element, and synthesizes a revised treatment plan based on the synthesis of the biofeedback element.

18. The device of claim 17, where a biofeedback from a patient is measure through electromyography.

19. The device of claim 17, where the number of heating coils is three.

20. The device of claim 19, where the Electromyography and electrical muscle stimulation band extends from an inside of the wand to an outer surface of the wand, where the Electromyography and electrical muscle stimulation band provides muscle stimulation and obtains biofeedback from the patient.
